(19) United States
(12) Patent Application Publication
(10) Pub. No.: US 2026/0115415 A1
(43) Pub. Date: Apr. 30, 2026

(54) RADIATION CONTAINMENT COMPONENTS, SEALING ASSEMBLIES, AND METHODS OF USE

(71) Applicant: BARD PERIPHERAL VASCULAR, INC., Franklin Lakes, NJ (US)

(72) Inventors: Christopher D. DROBNIK, Wauconda, IL (US); Angela JENSEN, Phoenix, AZ (US); Brandon D. SIMMONS, Tempe, AZ (US); Amanda THYSTRUP, Phoenix, AZ (US); Ryan T. TOOLEY, Tempe, AZ (US); Mark Nicholas WRIGHT, Coventry, RI (US)

(73) Assignee: BARD PERIPHERAL VASCULAR, INC., Franklin Lakes, NJ (US)

(21) Appl. No.: 18/867,388

(22) PCT Filed: May 25, 2022
(86) PCT No.: PCT/US2022/030849

§ 371 Date: Nov. 19, 2024

(51) Int. Cl. A61M 25/00; A61N 5/10; G21F 5/018

(52) U.S. Cl. CPC A61M 25/002; A61N 5/1002; A61N 5/1007; G21F 5/018; A61M 2025/0042; A61M 25/0097; A61N 2005/1021; A61N 2005/1089; A61N 2005/1094

(57) ABSTRACT

Radiation sealing assemblies and methods of use of a radiation containment component for sealing a microcatheter used to deliver a mixed particulate from a particulate delivery device for disposal, which may include a proximal end and a distal end disposed opposite the proximal end. The proximal end is configured to connect to and cover a distal portion of a delivery line connector of the particulate delivery device, the delivery line connector configured to receive the mixed particulate from the particulate delivery device. The distal end is configured to be disposed over and contain the microcatheter connected to the delivery line connector after use, and the distal end is configured to seal together to contain the microcatheter prior to disposal.

### TECHNICAL FIELD

The present disclosure generally relates to components of medical devices for treating cancer, and more particularly to radiation containment components of medical devices configured and operable to assist with delivery of radioactive compounds to a treatment area within a patient's body in procedures such as transarterial radioembolization.

### BACKGROUND

In cancer treatments involving radiation therapy, inadvertent or excess exposure to radiation from radioactive therapeutic agents can be harmful and potentially lethal to patients or medical personnel. Accordingly, medical instruments for radiation therapies must be configured to localize the delivery of radioactive material to a particular area of the patient's body while shielding others from unnecessarily being exposed to radiation.

Transarterial Radioembolization is a transcatheter intra-arterial procedure performed by interventional radiology and is commonly employed for the treatment of malignant tumors. During this medical procedure, a microcatheter is navigated into a patient's liver where radioembolizing microspheres loaded with a radioactive compound, such as yttrium-90 (⁹⁰Y), are delivered to the targeted tumors. The microspheres embolize blood vessels that supply the tumors while also delivering radiation to kill tumor cells. Generally, a clinician or patient may be at risk from radiation emitted from the delivery.

Accordingly, a need exists for components of a medical device configured and operable to shield from such radiation when delivering the radioactive compound to the patient's body.

### SUMMARY

In accordance with an embodiment of the disclosure, a radiation containment component for sealing a microcatheter used to deliver a mixed particulate from a particulate delivery device for disposal comprises a proximal end and a distal end disposed opposite the proximal end. The proximal end is configured to connect to and cover a distal portion of a delivery line connector of the particulate delivery device, the delivery line connector configured to receive the mixed particulate from the particulate delivery device. The distal end is configured to be disposed over and contain the microcatheter connected to the delivery line connector after use, and the distal end is configured to seal together to contain the microcatheter prior to disposal.

In another embodiment, a radiation sealing assembly for sealing and disposal comprising a particulate delivery device comprising a delivery line connector, a base connector, a microcatheter, and a radiation containment component. The microcatheter is used to deliver a mixed particulate from the particulate delivery device, the microcatheter configured to be connected to the base connector and the delivery line connector to deliver the mixed particulate, and the microcatheter configured to be disconnected from the base connector and connected to the delivery line connector after use. The radiation containment component comprises a proximal end and a distal end disposed opposite the proximal end. The proximal end is configured to connect to and cover a distal portion of the delivery line connector of the particulate delivery device, the delivery line connector configured to receive the mixed particulate from the particulate delivery device. The distal end is configured to be disposed over and contain the microcatheter connected to the delivery line connector after use. The distal end is configured to seal together to contain the microcatheter prior to disposal.

In yet another embodiment, a method for sealing and disposal of a microcatheter used to deliver a mixed particulate from a particulate delivery device comprising connecting a delivery line connector of the particulate delivery device to the microcatheter, connecting the microcatheter to a base connector, delivering the mixed particulate from the particulate delivery device through the microcatheter and the base connector, and disconnecting the microcatheter from the base connector after use. The method further comprises disposing a proximal end of a radiation containment component over a distal portion of the delivery line connector of the particulate delivery device such that the proximal end does not move in a distal direction, extending a distal end of the radiation containment component over to contain the microcatheter connected to the delivery line connector after use, and sealing the distal end together to contain the microcatheter prior to disposal.

These and additional features provided by the embodiments described herein will be more fully understood in view of the following detailed description, in conjunction with the drawings.

### BRIEF DESCRIPTION OF THE DRAWINGS

FIG. 1 is a perspective view of a delivery device including a protective shield and a vial sled, according to one or more embodiments shown and described herein;

FIG. 2 is a cross-sectional view of the vial sled of FIG. 1 according to one or more embodiments shown and described herein, the cross-section along line 2-2 of FIG. 1;

FIG. 3 is a perspective view of a vial assembly including an engagement head, according to one or more embodiments shown and described herein;

FIG. 4 is a partial cross-sectional view of the vial assembly of FIG. 4, the cross-section taken along line 4-4 of FIG. 3;

FIG. 5 is a perspective view of the vial sled of FIG. 1 with the vial assembly of FIG. 3 received therein, with a series of delivery lines coupled to the vial sled according to one or more embodiments shown and described herein;

FIG. 6 is a schematic side view of a radiation containment component in a folded position, according to one or more embodiments shown and described herein;

FIG. 7 is a schematic side view of the radiation containment component of FIG. 6 in the folded position disposed on a delivery line connector of a delivery device;

FIG. 8 is a schematic side view of the radiation containment component of FIG. 7 in an unfolded position being distally pulled over a microcatheter and approaching a base sheath receiving a distal end of the microcatheter;

FIG. 9 is a schematic side view of the radiation containment component of FIG. 7 in an unfolded position further distally pulled over the microcatheter and the base sheath receiving the distal end of the microcatheter; and

FIG. 10 is a schematic side view of the radiation containment component of FIG. 9 in an unfolded and sealed position for disposal and disposed and sealed over the distal end of the microcatheter removed from the base sheath.

### DETAILED DESCRIPTION

Reference will now be made in detail to various embodiments of delivery devices for administering radioactive compounds to a patient, examples of which are illustrated in the accompanying drawings. Whenever possible, the same reference numerals will be used throughout the drawings to refer to the same or like parts. Directional terms as used herein—for example up, down, right, left, front, back, top, bottom, distal, and proximal—are made only with reference to the figures as drawn and are not intended to imply absolute orientation.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

Unless otherwise expressly stated, it is in no way intended that any method set forth herein be construed as requiring that its steps be performed in a specific order, nor that with any apparatus specific orientations be required. Accordingly, where a method claim does not actually recite an order to be followed by its steps, or that any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred, in any respect. This holds for any possible non-express basis for interpretation, including: matters of logic with respect to arrangement of steps, operational flow, order of components, or orientation of components; plain meaning derived from grammatical organization or punctuation, and; the number or type of embodiments described in the specification.

Unless otherwise defined, all technical and scientific terms used herein have the same meaning as commonly understood by one of ordinary skill in the art to which the disclosure belongs. The terminology used in the description herein is for describing particular embodiments only and is not intended to be limiting. As used in the specification and appended claims, the singular forms “a,” “an,” and “the” are intended to include the plural forms as well, unless the context clearly indicates otherwise.

As used herein, the terms “horizontal,” “vertical,” “distal” and “proximal” are relative terms only, are indicative of a general relative orientation only, and do not necessarily indicate perpendicularity. These terms also may be used for convenience to refer to orientations used in the figures, which orientations are used as a matter of convention only and are not intended as characteristic of the devices shown. The present disclosure and the embodiments thereof to be described herein may be used in any desired orientation. Moreover, horizontal and vertical walls need generally only be intersecting walls, and need not be perpendicular. As used herein, the singular forms “a,” “an” and “the” include plural referents unless the context clearly dictates otherwise. Thus, for example, reference to “a” component includes aspects having two or more such components, unless the context clearly indicates otherwise.

In embodiments described herein, a particulate material delivery assembly may include a radioembolization delivery device. A radioembolization delivery device comprises a medical device configured to deliver radioactive compounds to a treatment area within a patient's body in procedures such as transarterial radioembolization. The radioactive compounds may be a mixed solution of saline and radioactive microspheres (i.e., a particulate) mixed in a vial of a vial assembly. The needle may include one or more ports as an outlet to inject fluid (i.e., saline), such as from a syringe or catheter line, into a vial including the radioactive microspheres to generate the mixed solution and as an inlet to deliver the mixed solution to the patient.

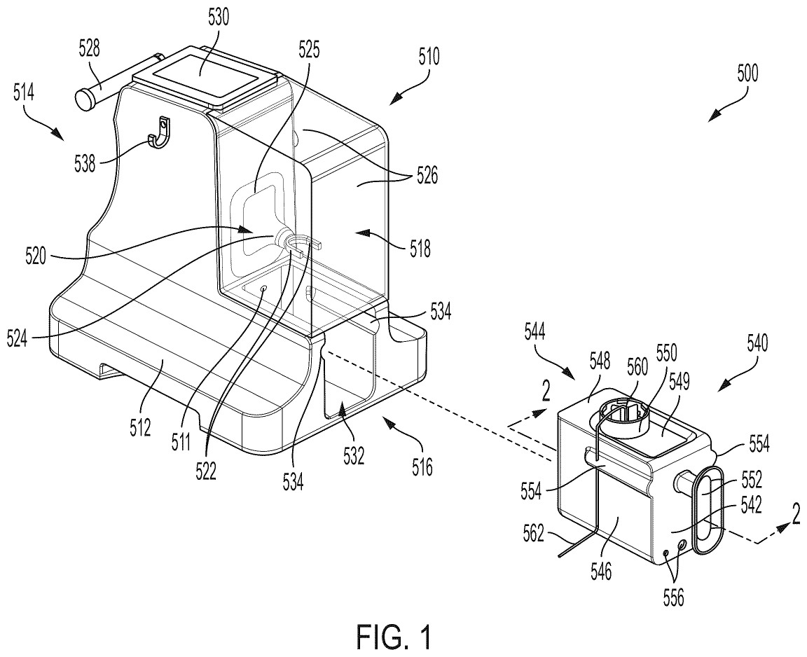

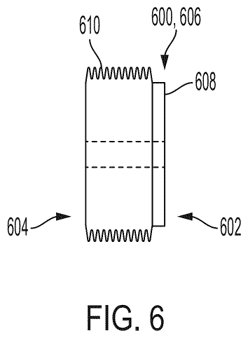

FIGS. 1-5 described below are directed to an embodiment of a delivery device 500 to deliver a particulate, and FIGS. 6-10 described in greater detail further below are directed to embodiments of one or more components of the delivery device 500 as described herein assist with shielding from radiation emitted from the particulate as secondary radiation containment components 600. The radiation containment components 600 as described herein and in greater detail further below with respect to FIGS. 6-10 may further aid to prevent the release the hazardous materials when disposing of a used delivery device connected to a microcatheter. Such hazardous materials may include chemotherapy drugs, radioactive substances, blood and/or other biohazards that may cause health hazards if released into a surrounding environment. The radiation containment components 600 may be utilized to dispose of potentially contaminated accessory devices as described herein used during radioembolization procedures, delivery of potentially hazardous drugs, or combinations thereof. For example, when removing a microcatheter 720 or delivery sheath from a patient after delivery, a potential exists for a leak that could contaminate an environment and be hazardous to a user, such a physician and/or surrounding users or health personnel. The radiation containment components 600 described herein and in greater detail further below with respect to FIGS. 6-10 may be placed on a delivery line connector 710 of the delivery device 500 prior to connection of the delivery line connector 710 to a hub of a microcatheter 720. After a final injection is a made, the user may be slide the radiation containment component 600 toward the hub of the microcatheter 720 and then pull the delivery line connector 710 to further pull the microcatheter 720 from a base connector (such as a base sheath 730 described herein) into the radiation containment component 600, which may then be sealed for disposal as described herein. The user may maintain a distal end 604 of the radiation containment component 600 over a hub of the base connector to ensure the microcatheter 720 is contained upon removal from the hub of the base connector. Once the entire microcatheter 720 is contained in the radiation containment component 600, the user may seal a corresponding end of the radiation containment component 600 with, for example, an adhesive on the distal end 604 of the radiation containment component 600. The user may then dispose of the particulate delivery device 500 as contained by the radiation containment component 600 as described herein based on hazardous disposal procedures. For example, the user may disposed of the contained particulate delivery device 500 using sterile towels wrapped around the contained particulate delivery device 500, which towels may further be rolled and placed into a waste container for disposal. In embodiments, the radiation containment component 600 may be placed on the delivery line connector 710 prior to delivery of the mixed particulate. In other embodiments, the radiation containment component 600 may be split down an inner location such as a center to be separately wrapped around the particulate delivery device 500 and the microcatheter 720 such that installation prior to user is not required.

In some embodiments, as described in greater detail below, the delivery device 500 is a radioembolization delivery device, the particulate is a plurality of radioembolization beads, the fluid is a saline solution, and the resulting mixed fluid (e.g., the mixed fluid solution) is a radioembolization beads-saline solution. The needle 559 may be configured to deliver the radioembolization beads-saline solution as the mixed fluid solution through the radioembolization delivery device, such as upon actuation of the vial engagement mechanism 520 in the positive pressure direction. In some embodiments, the fluid is a contrast-saline solution including a contrast agent, and the resulting mixed fluid (e.g., the mixed fluid solution) is a radioembolization beads-contrast-saline solution. The needle 559 may be configured to deliver the radioembolization beads-contrast-saline solution as the mixed fluid solution through the radioembolization delivery device. In some embodiments, the delivery device 500 is a chemoembolization delivery device, the particulate is a plurality of chemoembolization beads, and the mixed fluid solution is a beads-saline solution or a beads-contrast-saline solution.

### I. Mechanical Delivery Device with Removable Sled Assembly

FIGS. 1-5 show an embodiment of a delivery device 500 that is configured and operable to deliver a radioactive material (e.g., radioembolizing beads) while reducing radioactive emissions during use of the delivery device 500. The delivery device 500 may operate as described in International PCT App. No. PCT/2019/033001, filed May 17, 2019, the entirety of which is incorporated herein, except with respect to radiation shield components as described in greater detail below with respect to FIGS. 6-10 and in one or more embodiments herein.

Referring initially to FIG. 1, the delivery device 500 comprises a console assembly 510, which includes a console. The delivery device 500 may include a sled assembly 540 that is operable to transition between a coupled state and decoupled state relative to the console assembly 510. The console assembly 510 of the delivery device 500 comprises a base 512 defined by and extending between a proximal end 514 and a distal end 516. The proximal end 514 of the base 512 includes a handle (delivery handle) 528 movably coupled to the console assembly 510 and an interface display 530 positioned on the console assembly 510.

The proximal end 514 of the base 512 further includes an attachment device 538 that is configured to securely retain an external device to the base 512 of the console assembly 510. The attachment device 538 is operable to facilitate an attachment of a complimentary device to the console assembly 510 for use with the delivery device 500 during a procedure.

Still referring to FIG. 1, the distal end 516 of the console assembly 510 defines a vial containment region 518 that is sized and shaped to receive a vial assembly 580 therein, as will be described in greater detail herein. The console assembly 510 further includes a vial engagement mechanism 520 extending from the base 512 adjacent to the distal end 516. In particular, the vial engagement mechanism 520 extends laterally outward from the base 512 of the console assembly 510 toward the distal end 516. The vial engagement mechanism 520 is positioned within the vial containment region 518 of the console assembly 510 and is movably coupled to the handle 528. In particular, the handle 528 of the console assembly 510 is operable to move, and in particular translate, the vial engagement mechanism 520 within the vial containment region 518 in response to an actuation of the handle 528.

The console assembly 510 includes a mechanical assembly disposed within the base 512 that is configured and operable to convert a manual motion of the handle 528 to a corresponding linear displacement of the vial engagement mechanism 520. In the present example, the mechanical assembly is coupled to the handle 528 and the vial engagement mechanism 520 such that selective actuation of the handle 528 at the proximal end 514 causes a simultaneous actuation of the vial engagement mechanism 520 at the distal end 516.

The sled cavity 532 is sized and shaped to receive the sled assembly 540 therein. As will be described in greater detail herein, the sled assembly 540 is configured to store and administer therapeutic particles (e.g., radioactive beads, microspheres, medium) therethrough. In particular, the sled assembly 540 is configured to partially receive a vial assembly 580 therein for administering the therapeutic particles from the delivery device 500 and to a patient during a procedure.

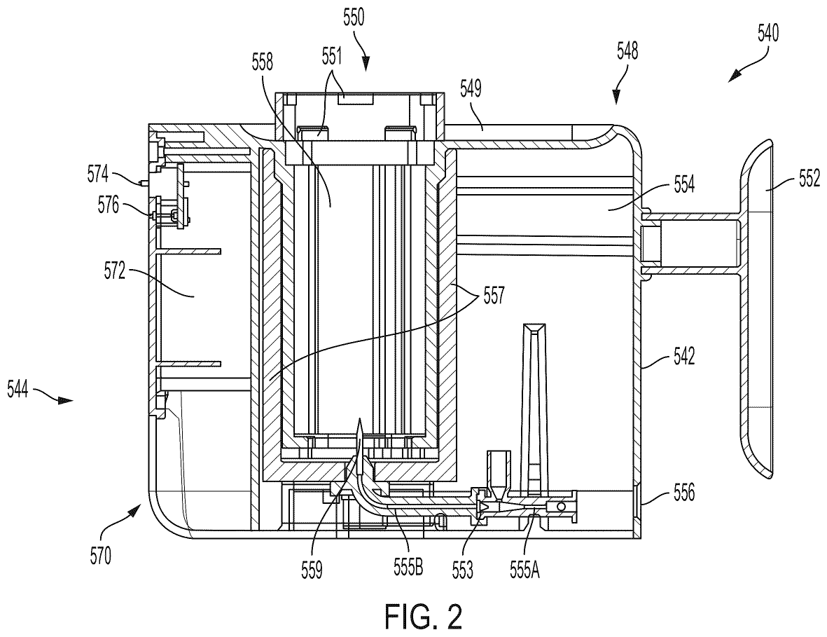

In embodiments, and referring to FIG. 2, a flow sensor of the delivery device 500 may be positioned in-line with the tubing set of the delivery device 500, and in particular the needle 559, the manifolds 555A, 555B, and/or one or more of the ports 556, and may be configured to measure an amount of fluid (e.g., suspension liquid after the therapeutic particles have effectively mixed with the fluid medium) that passes thereby. Referring back to FIG. 1, the vial engagement mechanism 520 comprises a pair of lever arms 522 extending outwardly from a neck 524 of the vial engagement mechanism 520, with the neck 524 extending laterally outward from the base 512 of the console assembly 510. The neck 524 of the vial engagement mechanism 520 is disposed within a protective cover 525 such that only the pair of lever arms 522 of the vial engagement mechanism 520 extends through the protective cover 525. The protective cover 525 is operable to shield one or more internal components of the console assembly 510 from an exterior of the console assembly 510, and in particular from the vial containment region 518.

The pair of lever arms 522 is simultaneously movable with the neck 524 of the vial engagement mechanism 520 in response to an actuation of the handle 528 of the console assembly 510. Further, the pair of lever arms 522 are fixed relative to one another such that a spacing formed between the pair of lever arms 522 is relatively fixed. The pair of lever arms 522 of the vial engagement mechanism 520 is configured to securely engage the vial assembly 580 therebetween, and in particular within the spacing formed by the pair of lever arms 522. Accordingly, the vial engagement mechanism 520 is operable to securely attach the vial assembly 580 to the console assembly 510 at the vial containment region 518. Although the vial engagement mechanism 520 is shown and described herein as including a pair of lever arms 522, it should be understood that the vial engagement mechanism 520 may include various other structural configurations suitable for engaging the vial assembly 580. In a non-limiting example, the vial engagement mechanism 520 may include one or more magnets configured to engage with one or more corresponding magnets on the vial assembly.

Still referring to FIG. 1, the console assembly 510 further includes a safety shield 526 secured to the distal end 516 of the base 512 along the vial containment region 518. In particular, the safety shield 526 is a protective covering that is sized and shaped to enclose the vial containment region 518 of the console assembly 510 when secured thereon. The safety shield 526 is selectively attachable to the distal end 516 of the base 512 and is formed of a material that is configured to inhibit radioactive emissions from one or more radioactive doses stored within the vial containment region 518.

The distal end 516 of the console assembly 510 further includes a sled cavity 532 that is sized and shaped to receive the sled assembly 540 therein. The sled cavity 532 includes one or more or a pair of alignment features 534 extending therein, with the alignment features 534 sized and shaped to correspond with complimentary alignment features of the sled assembly 540 (e.g., alignment ribs 554) to thereby facilitate a coupling of the sled assembly 540 with the base 512 of the console assembly 510 within the sled cavity 532.

Still referring to FIG. 1, the sled assembly 540 is configured to partially receive a vial assembly 580 therein for administering therapeutic particles (e.g., radioactive fluid medium) from the delivery device 500 and to a patient. In particular, the sled assembly 540 comprises a distal end 542 and a proximal end 544 with a pair of sidewalls 546 extending therebetween. The distal end 542 of the sled assembly 540 includes a handle 552 extending proximally therefrom. The handle 552 is configured to facilitate movement of the sled assembly 540, and in particular, an insertion of the sled assembly 540 into the sled cavity 532 of the console assembly 510. The distal end 542 further includes one or more ports 556 for coupling one or more delivery lines (i.e., tubing) to the sled assembly 540. With the one or more delivery lines further be coupled to one or more external devices at an end of the line opposite of the ports 556, the ports 556 effectively serve to fluidly couple the sled assembly 540 to the one or more external devices via the delivery lines connected thereto. The pair of sidewalls 546 of the sled assembly 540 includes at least one alignment rib 554 extending laterally outward therefrom, where the alignment ribs 554 are sized and shaped to correspond with and mate to the pair of alignment features 534 of the console assembly 510. Accordingly, the pair of alignment ribs 554 are configured to facilitate an alignment and engagement of the sled assembly 540 with the console assembly 510 when the proximal end 544 is slidably received within the sled cavity 532 of the base 512.

The sled assembly 540 further includes a top surface 548 extending from the distal end 542 and the proximal end 544 and positioned between the pair of sidewalls 546. The top surface 548 of the sled assembly includes a recessed region 549 and a locking system 550. The recessed region 549 is sized and shaped to form a recess and/or cavity along the top surface 548, where the recessed region 549 is capable of receiving and/or collecting various materials therein, including, for example, leaks of various fluid media during use of the delivery device 500. The locking system 550 of the sled assembly 540 forms an opening along the top surface 548 that is sized and shaped to receive one or more devices therein, such as a priming assembly 560 and a vial assembly 580. In some embodiments, the sled assembly 540 comes preloaded with the priming assembly 560 disposed within the locking system 550. The priming assembly 560 includes a priming line 562 extending outwardly from the locking system 550 of the sled assembly 540. The priming assembly 560 connects the priming line 562 to needle 559 and manifolds 555A and 555B and serves to purge the delivery device 500, including the manifolds 555A and 555B, of air prior to utilizing the delivery device 500 in a procedure.

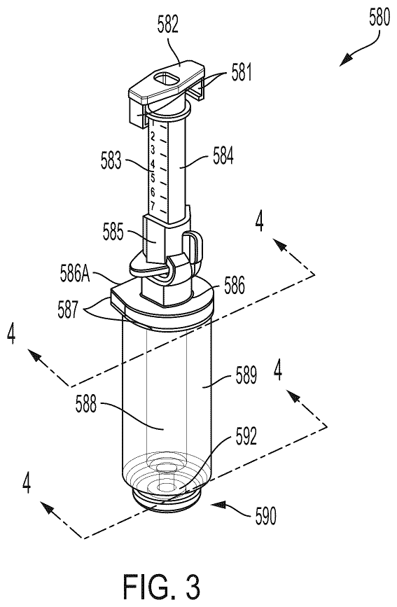

Referring now to FIG. 2, the locking system 550 includes an annular array of projections 551 extending outwardly therefrom, and in particular, extending laterally into the aperture formed by the locking system 550 along the top surface 548. The annular array of projections 551 are formed within an inner perimeter of the locking system 550 and extend along at least two sequentially-arranged rows. In embodiments, a single row may be used. The annular array of projections 551 included in the locking system 550 are configured to engage a corresponding locking feature 586 of the vial assembly 580 (See FIG. 3) to thereby securely fasten the vial assembly 580 to the sled assembly 540. It should be understood that the multiple rows of projections 551 of the locking system 550 serve to provide a double-locking system to ensure the sled assembly 540, and in particular a needle 559 of the sled assembly 540, is securely maintained through a septum 592 of the vial assembly 580 (See FIG. 3) during use of the delivery device 500 in a procedure.

The sled assembly 540 further includes a vial chamber 558 that is sized and shaped to receive the priming assembly 560 and the vial assembly 580 therein, respectively. In other words, the vial chamber 558 is sized to individually receive both the priming assembly 560 and the vial assembly 580 separate from one another. The vial chamber 558 is encapsulated around a protective chamber or shield 557 disposed about the vial chamber 558. The protective shield 557 is formed of a material configured to inhibit radioactive emissions from extending outwardly from the vial chamber 558, such as, for example, a metal or plastic. Additionally, the sled assembly 540 includes a needle extending through the protective shield 557 and into the vial chamber 558 along a bottom end of the vial chamber 558. The needle 559 is fixedly secured relative to the vial chamber 558 such that any devices received through the aperture of the locking system 550 and into the vial chamber 558 are to encounter and interact with the needle 559 (e.g., the priming assembly 560, the vial assembly 580, and the like).

Still referring to FIG. 2, the needle 559 is coupled to a distal manifold 555A and a proximal manifold 555B disposed within the sled assembly 540, and in particular the manifold 555A, 555B is positioned beneath the vial chamber 558 and the protective shield 557. The proximal manifold 555B is fluidly coupled to the needle 559 and the distal manifold 555A is fluidly couplable to one or more delivery lines via the one or more ports 556 of the sled assembly 540. The proximal manifold 555B is in fluid communication with the distal manifold 555A through a one-way check valve 553 disposed therebetween.

Accordingly, the proximal manifold 555B is in fluid communication with the one or more ports 556 via the distal manifold 555A, however, the one or more ports 556 are not in fluid communication with the proximal manifold 555B due to a position of the one-way check valve 553 disposed between the manifolds 555A, 555B. Thus, the needle 559 is in fluid communication with the one or more delivery lines and/or devices coupled to the sled assembly 540 at the one or more ports 556 via the manifolds 555A, 555B secured therebetween. The one or more ports 556 of the sled assembly 540 may be coupled to a bag (e.g., saline bag), a syringe, a catheter, and/or the like via one or more delivery lines coupled thereto. In other embodiments, the needle 559 may be a cannula, catheter, or similar mechanism through which to inject and receive fluid and/or a solution as described herein.

Still referring to FIG. 2, the sled assembly 540 includes a removable battery pack 570 coupled to the sled assembly 540 along the proximal end 544. The removable battery pack 570 comprises a battery 572, electrical contacts 574, and a removable tab 576. The battery 572 of the delivery device 500 is isolated from one or more fluid paths and radiation sources due to a location of the battery 572 in the removable battery pack 570.

The electrical contacts 574 of the removable battery pack 570 extend outwardly from the removable battery pack 570 and are operable to contact against and interact with corresponding electrical contacts 511 of the console assembly 510 (See FIG. 1) when the sled assembly 540 is coupled to the base 512 at the sled cavity 532. Accordingly, the removable battery pack 570 is operable to provide electrical power to the delivery device 500, and in particular the console assembly 510, when the sled assembly 540 is coupled to the console assembly 510.

Additionally, as will be described in greater detail herein, in some embodiments the locking system 550 may include at least one planar wall relative to a remaining circular orientation of the locking system 550. In this instance, an aperture formed by the locking system 550 through the top surface 548 of the sled assembly 540 is irregularly-shaped, rather than circularly-shaped as shown and described above. In this instance, the vial assembly 580 includes a locking feature 586 that has a shape and size that corresponds to the locking system 550, and in particular the at least one planar wall such that the vial assembly 580 is received within the sled assembly 540 only when an orientation of the vial assembly 580 corresponds with an alignment of the locking feature 586 and the locking system 550. In other words, a corresponding planar wall 586A of the locking feature 586 (See FIG. 3) must be aligned with the planar wall of the locking system 550 for the vial assembly 580 to be receivable within an aperture formed by the locking system 550 of the sled assembly 540.

Referring now to FIG. 3, the vial assembly 580 of the delivery device 500 is depicted. The vial assembly 580 comprises an engagement head 582, a plunger 584, a locking feature 586, and a vial body 589. In particular, the engagement head 582 of the vial assembly 580 is positioned at a terminal end of the plunger 584 opposite of the locking feature 586 and the vial body 589. The engagement head 582 includes a pair of arms 581 extending laterally outward relative to a longitudinal length of the plunger 584 extending downwardly therefrom. In the present example, the engagement head 582 is integrally formed with the plunger 584, however, it should be understood that in other embodiments the engagement head 582 and the plunger 584 may be separate features fastened thereto. In either instance, the engagement head 582 and the plunger 584 is movable relative to the locking feature 586 and the vial body 589 such that the engagement head 582 and the plunger 584 are slidably translatable through the locking feature 586 and the vial body 589. In particular, as will be described in greater detail herein, the plunger 584 may translate into and out of an internal chamber 588 of the vial body 589 in response to a linear translation of the vial engagement mechanism 520 when the engagement head 582 is secured to the pair of lever arms 522.

The plunger 584 includes a plurality of indicia and/or markings 583 positioned along a longitudinal length of the plunger 584. The plurality of markings 583 is indicative of a relative extension of the engagement head 582 and the plunger 584 from the locking feature 586 and the vial body 589. As briefly noted above, the engagement head 582 is configured to attach the vial assembly 580 to the vial engagement mechanism 520. In particular, the pair of arms 581 of the engagement head 582 are sized and shaped to couple with the pair of lever arms 522 of the vial engagement mechanism 520 when the vial assembly 580 is received within the sled assembly 540 and the sled assembly is inserted into the sled cavity 532 of the console assembly 510. As will be described in greater detail herein, the pair of lever arms 522 are received between the pair of arms 581 of the engagement head 582 and the plunger 584 in response to a predetermined translation force applied to the vial engagement mechanism 520. The engagement head 582 and the plunger 584 may be formed of various materials, including, but not limited to, a metal, plastic, and/or the like.

Still referring to FIG. 3, the vial assembly 580 further includes a safety tab 585 coupled to the plunger 584 relatively above the locking feature 586 and below the engagement head 582 such that the safety tab 585 is positioned along the longitudinal length of the plunger 584. The safety tab 585 may be formed of various materials, such as, for example, a plastic, and is preassembled onto the vial assembly 580 prior to a use of the delivery device 500. The safety tab 585 is removably fastened to the plunger 584 and inhibits the plunger 584 from translating relative to the vial body 589. In particular, the safety tab 585 abuts against the locking feature 586 in response to an application of linear force onto the plunger 584 to translate the plunger 584 relatively downward into the vial body 589. In this instance, the safety tab 585 is configured to inhibit an inadvertent movement of the plunger 584, and in response, an inadvertent delivery of a fluid media stored within the internal chamber 588 of the vial body 589 (e.g., therapeutic particles, radioembolizing beads). As will be described in greater detail herein, the safety tab 585 is selectively disengaged from the plunger 584 in response to a coupling of the vial assembly 580 with the vial engagement mechanism 520, and in particular an engagement of the pair of lever arms 522 with the engagement head 582.

Referring back to FIG. 3, the locking feature 586 extends about a top end of the vial body 589. In the present example, the locking feature 586 of the vial assembly 580 comprises a bushing that defines a lateral edge 587 extending laterally outward along an outer perimeter of the locking feature 586. The lateral edge 587 of the locking feature 586 is sized and shaped to engage the annular array of projections 551 of the locking system 550 when the vial assembly 580 is received within the vial chamber 558 of the sled assembly 540. As will be described in greater detail herein, the locking feature 586, and in particular the lateral edge 587 of the locking feature 586, is configured to securely fasten the vial assembly 580 to the locking system 550 to inhibit removal of the vial body 589 from the vial chamber 558 of the sled assembly 540 during use of the delivery device 500 in a procedure. In some embodiments, as briefly described above, the locking feature 586 includes at least one planar wall 586A such that the locking feature 586 comprises an irregular-profile. The at least one planar wall 586A is configured to correspond to the planar wall 550A of the locking system 550 such that an alignment of the planar walls 550A, 586A is required for the vial assembly 580 to be received through an aperture formed by the locking system 550.

Still referring to FIG. 3, the vial body 589 extends downwardly relative from the locking feature 586 and has a longitudinal length that is sized to receive at least a portion of a longitudinal length of the plunger 584 therein. Accordingly, in some embodiments a longitudinal length of the plunger 584 exceed a longitudinal length of the vial body 589 such that a translation of the plunger 584 into the internal chamber 588 of the vial body 589 causes a fluid media stored therein to be transferred outward from the vial body 589. As will be described in greater detail herein, a translation of the plunger 584 through the internal chamber 588 of the vial body 589 provides for an administration of a fluid media stored within the vial body 589 outward from the vial assembly 580. The vial body 589 may be formed of various materials, including, for example, a thermoplastic polymer, copolyester, polycarbonate, a biocompatible plastic, polysulfone, ceramics, metals, and/or the like.

The vial body 589 is of the present example is formed of a material that is configured to inhibit radioactive emissions from a fluid media stored within the internal chamber 588 of the vial body 589. For example, the vial body 589 may be formed of a plastic, such as polycarbonate, and have a width. A density and material composition of the vial body 589 may collectively inhibit beta radiation emission from electron particles stored within the internal chamber 588. In the present example, a chemical composition of the plastic of the vial body 589, along with the 9 mm wall thickness, provides a plurality of atoms disposed within the vial body 589 that are capable of encountering the electron particles generating beta radiation and reducing an emission of said radiation from the vial assembly 580. Accordingly, the vial assembly 580 allows an operator to handle the radioactive material stored within the vial body 589 without being exposed to beta radiation. It should be understood that various other materials and/or wall sections may be incorporated in the vial body 589 of the vial assembly 580 in other embodiments without departing from the scope of the present disclosure.

Still referring to FIG. 3, the vial body 589 of the vial assembly 580 is sealed at a first terminal end 598 by the locking feature 586. The vial assembly 580 further includes a cap 590 positioned at an opposing, terminal end of the vial body 589 opposite of the locking feature 586, such that the cap 590 seals a second terminal end of the vial body 589 of the vial assembly 580. Additionally, the vial assembly 580 includes a septum 592 positioned adjacent to the cap 590 and in fluid communication with a terminal end of the vial body 589 opposite of the locking feature 586. The septum 592 forms a seal against a terminal end of the vial body 589 and the cap 590 retains the septum 592 therein. The septum 592 may be formed of various materials, including, for example, an elastomer, silicon, bromobutyl elastomer, rubber, urethanes, and/or the like. The septum 592 is configured to provide an air-tight seal for the vial body 589 to thereby inhibit a release of a fluid media stored therein (e.g., radioembolizing beads). As will be described in greater detail herein, the septum 592 of the vial assembly 580 is configured to be punctured by the needle 559 of the sled assembly 540 when the vial assembly 580 is received within the vial chamber 558, thereby establishing fluid communication between the vial body 589 and the sled assembly 540. In other embodiments, the septum 592 may be omitted entirely for an alternative device, such as, for example, a valve system, needle injection port, and/or the like.

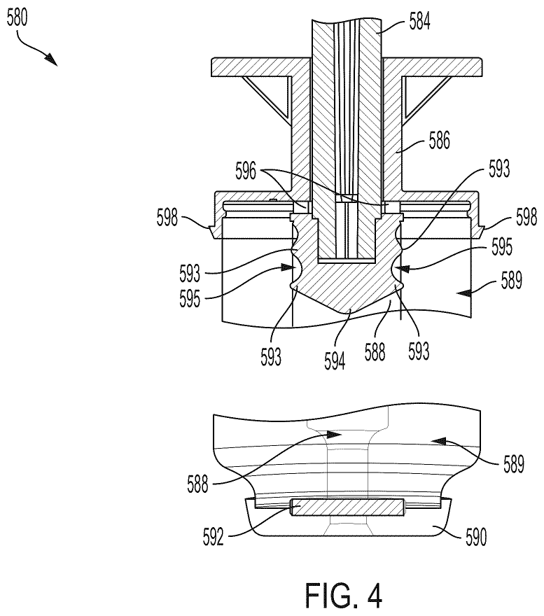

Referring to FIG. 4, the vial assembly 580 further includes a stopper 594 fixedly coupled to a terminal end of the plunger 584 opposite of the engagement head 582. In this instance, with the plunger 584 coupled to, and slidably translatable through, the internal chamber 588 of the vial body 589, the stopper 594 is effectively disposed within the vial body 589. Accordingly, it should be understood that the stopper 594 is sized and shaped in accordance with a size (e.g., a diameter) of the internal chamber 588 of the vial body 589. The stopper 594 is secured to the plunger 584 such that the stopper 594 is slidably translatable through the vial body 589 in response to a translation of the plunger 584 through the vial body 589. The stopper 594 is defined by two or more ribs 593 extending laterally outward and one or more troughs 595 defined between at least two ribs 593.

The stopper 594 is configured to form a liquid-seal against the internal chamber 588 of the vial body 589, and may be formed of a various polymers with a predetermined viscoelasticity. For example, in some embodiments the stopper 594 is formed of an elastomer, silicone, rubber, urethane, plastic, polyethylene, polypropylene, and/or the like. In this instance, the stopper 594 is operable to inhibit a fluid media stored within the vial body 589 from extending (i.e., leaking) past the stopper 594 and out of the vial body 589. In particular, the two or more ribs 593 of the stopper 594 abut against, and form a seal along, the internal chamber 588 of the vial body 589 to thereby inhibit a fluid media from passing beyond the ribs 593. The one or more troughs 595 formed between the two or more ribs 593 of the stopper 594 are configured to receive, and more specifically capture, any fluid media that may inadvertently extend (i.e., leak) beyond the ribs 593 of the stopper 594. Accordingly, the one or more troughs 595 serve as a safety mechanism of the vial assembly 580 to ensure a fluid media is maintained within the vial body 589 and not exposed beyond the vial assembly 580.

Still referring to FIG. 4, the two or more ribs 593 of the stopper 594 are additionally configured to push a fluid media stored within the vial body 589 in one or more directions therein (e.g., toward the cap 590) in response to a translation of the plunger 584. With the ribs 593 of the stopper 594 pressed against the internal chamber 588 of the vial body 589, translation of the plunger 584 provides for a translation of the ribs 593 against and along the internal chamber 588 of the vial body 589 such that any fluid media located in front (i.e., beneath) of the stopper 594 is effectively redirected within the vial body 589 in a direction of travel of the plunger 584 and the stopper 594. The vial assembly 580 further includes an annular washer 596 disposed within the vial body 589. In particular, the annular washer 596 is securely fixed to the plunger 584 adjacent to the stopper 594, which is secured to the plunger 584 at a terminal end opposite of the engagement head 582. Accordingly, the annular washer 596 is secured to the plunger 584 and disposed within the vial body 589 adjacent to the stopper 594. With the annular washer 596 secured to the plunger 584 adjacent to the stopper 594, the annular washer 596 is effectively disposed within the vial body 589.

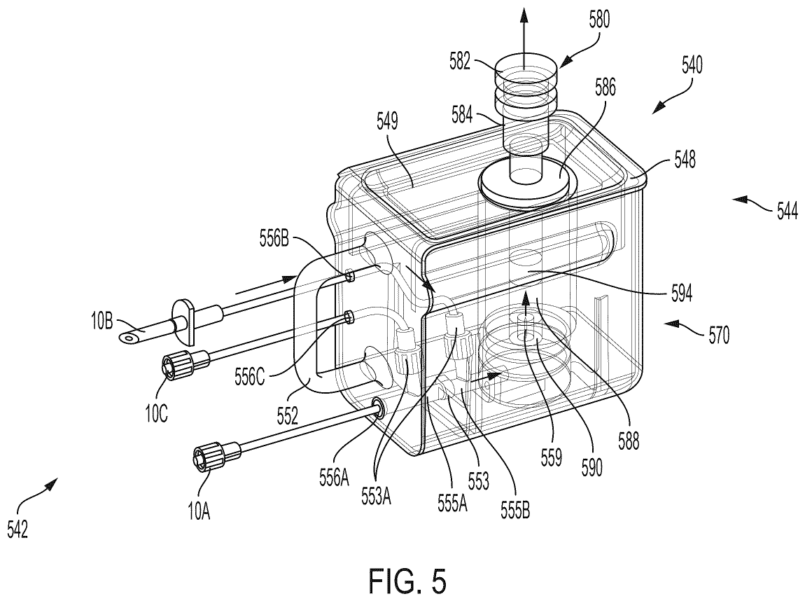

Referring now to FIG. 5, in response to determining that the battery 572 contains or other power source provides a sufficient amount of power, one or more delivery lines are coupled to the sled assembly 540 via the one or more ports 556. In particular, a dose delivery line 10A is coupled to the sled assembly 540 at a delivery port 556A, a contrast line 10B is coupled to the sled assembly 540 at a contrast port 556B, and a flushing line 10C is coupled to the sled assembly 540 at a flushing port 556C. An opposing end of the dose delivery line 10A is initially coupled to a fluid reservoir, such as, for example, a collection bowl. As will be described in greater detail herein, the dose delivery line 10A may be subsequently coupled to an external device, such as a catheter, once the sled assembly 540 has been effectively primed by a fluid medium via the contrast line 10B. An opposing end of the flushing line 10C is coupled to an external device, such as, for example, a syringe. With both the dose delivery line 10A and the flushing line 10C coupled to the sled assembly 540, the sled assembly 540 is flushed with a fluid medium (e.g., saline) from the syringe coupled to the flushing line 10C. In this instance, the fluid medium is injected through the flushing line 10C, into the distal manifold 555A of the sled assembly 540, and out of the sled assembly 540 through the dose delivery line 10A. Accordingly, the fluid medium is ultimately received at the collection bowl and disposed thereat by the dose delivery line 10A.

With the distal manifold 555A of the sled assembly 540 separated from the proximal manifold 555B by the one-way valve 553 disposed therebetween, the fluid medium flushed through the distal manifold 555A from the syringe (via the flushing port 556C) is prevented from passing through the proximal manifold 555B and the needle 559 coupled thereto. Rather, the fluid medium injected from the syringe and through the flushing line 10C is received at the flushing port 556C, passed through the distal manifold 555A in fluid communication with the flushing port 556C, and redirected by the one-way valve 553 towards the dose delivery port 556A that is coupled to the dose delivery line 10A. In this instance, the dose delivery line 10A receives and transfers the fluid medium to the collection bowl coupled thereto, such that the fluid medium is not directed beyond the one-way valve 553 and into the proximal manifold 555B that is in fluid communication with the needle 559.

The contrast line 10B is coupled to the sled assembly 540 at a contrast port 556B. An opposing end of the contrast line 10B is coupled to a fluid medium supply, such as, for example, a bag secured to the console assembly 510 via the attachment device 538. In the present example, the bag is a saline bag such that the fluid medium stored therein is saline. In this instance, with the sled assembly 540 including the priming assembly 560 positioned within the vial chamber 558 and the needle end 568 in fluid communication with the needle 559, a syringe is fluidly coupled to the priming line 562 of the priming assembly 560 and a plunger of the syringe is drawn back to pull saline through the contrast line 10B, the contrast port 556B, the sled assembly 540, the priming line 562 and into the syringe from the saline bag. The plunger of the syringe is thereafter pushed inwards to transfer the extracted saline back through the priming line 562, the central body 564, the elongated shaft 566, and the needle end of the priming assembly 560 such that the saline is received into the needle 559 of the sled assembly 540. Accordingly, the manifolds 555A, 555B of the sled assembly 540 are effectively primed with the saline from the syringe as the needle 559 that received the saline from the priming assembly 560 is in fluid communication with the manifolds 555A, 555B. With the manifolds 555A, 555B in further fluid communication with the dose delivery line 10A via the delivery port 556A, the saline is effectively distributed to the collection bowl coupled thereto.

Referring now to FIG. 5, the sled assembly 540 is coupled to one or more external devices via the one or more ports 556. In particular, the sled assembly 540 is fluidly coupled to a catheter (e.g., microcatheter) via the dose delivery line 10A that is coupled to the delivery port 556A of the sled assembly 540. In this instance, the catheter is in fluid communication with the sled assembly 540 via the dose delivery line 10A. Further, the sled assembly 540 is fluidly coupled to a contrast source, such as, for example, a saline bag secured to the console assembly 510 via the attachment device 538 (See FIG. 1). The sled assembly 540 is in fluid communication with the saline bag via a contrast line 10B coupled to the contrast port 556B of the sled assembly 540. In this instance, the saline bag is in fluid communication with the sled assembly 540 via the contrast line 10B secured to the contrast port 556B.

The contrast port 556B is in fluid communication with the proximal manifold 555B while the delivery port 556A is in fluid communication with the distal manifold 555A. As will be described in greater detail herein, saline from the saline bag may be withdrawn through the needle 559 of the sled assembly 540 and into the vial body 589 of the vial assembly 580 as the contrast port 556B is coupled to the proximal manifold 555B, rather than the distal manifold 555A which is separated from the proximal manifold 555B by the one-way check valve 553 disposed therebetween.

Referring again to FIGS. 1 and 3, with the vial assembly 580 securely coupled to the sled assembly 540, the sled assembly 540 is coupled to the console assembly 510 by translating the distal end 542 of the sled assembly 540 toward and into the distal end 516 of the console assembly 510. In particular, the distal end 542 of the sled assembly 540 is directed into the sled cavity 532 of the console assembly 510 by aligning the alignment ribs 554 of the sled assembly 540 with the alignment features 534 of the console assembly 510. Once the proximal end 544 and the distal end 542 of the sled assembly 540 are fully seated within the sled cavity 532 of the console assembly 510, the electrical contacts 574 (FIG. 2) of the removable battery pack 570 interact with corresponding electrical contacts 511 (FIG. 1) of the console assembly 510. In this instance, power from the battery 572 is transmitted to the console assembly 510 via the electrical contacts 574, thereby activating the console assembly 510 of the delivery device 500. In this instance, the interface display 530 of the console assembly 510 is activated to display pertinent, real-time information relating to the delivery device 500 during a procedure.

Referring again to FIG. 5, as the vial engagement mechanism 520 and the plunger 584 are simultaneously translated within the vial containment region 518, a negative pressure is generated within the internal chamber 588 of the vial body 589 due to a retraction of the stopper 594. In this instance, with the saline bag coupled to the sled assembly 540 via the contrast line 10B and the contrast port 556B, saline from the saline bag is pulled into the internal chamber 588 of the vial body 589 through the proximal manifold 555B and the needle 559. Accordingly, with the vial body 589 being preloaded with a radioactive fluid media (e.g., radioembolizing microspheres), the saline is effectively mixed with the radioactive fluid media within the vial body 589 as the plunger 584 is retracted from the internal chamber 588 and the negative pressure is generated through the delivery device 500.

The sled assembly 540 further includes one-way check valves 553A in-line with the contrast line 10B and the flushing line 10C. In particular, the one-way check valves 553A are configured to permit fluid communication from the contrast port 556B and the flushing port 556C into the manifolds 555A, 555B, and further configured to prevent fluid communication from the manifolds 555A, 555B to the contrast port 556B and the flushing port 556C. Accordingly, it should be understood that the dose delivered from the vial body 589 to the manifold 555A, 555B is incapable of being directed into the contrast line 10B or the flushing line 10C due to the one-way check valves 553A positioned therein. Thus, the dose is directed to the dose delivery port 556A and received at the catheter fluidly coupled thereto by the dose delivery line 10A. In other words, the one-way check valves 553A prevent a backflow of fluid into the sled assembly 540 and/or the vial assembly 580 coupled thereto.

### II. Radiation Containment Embodiments

As briefly noted above, the delivery device 500 described herein may include a radiation containment component, embodiments of which are described in greater detail below with respect to FIGS. 6-10. FIGS. 6-10 show an embodiment of a radiation containment component 600 for disposal or disposed between a delivery line connector 710 (e.g., the dose delivery line 10A) of a particulate delivery device 500 and a microcatheter 720, as described in greater detail below. Thus, FIGS. 6-10 reflect embodiments of a radiation containment component 600 such as a flexible bag for sealing over a microcatheter 720 used to deliver a mixed particulate from a particulate delivery device 500 for disposal, the mixed particulate delivered from a delivery line connector 710 of the particulate delivery device 500 to the microcatheter 720, particularly after use such that a distal end 728 of the microcatheter 720 is sealed and the delivery line connector 710 connected to the microcatheter 720 after use is ready for disposal (e.g., to be thrown away in a hazardous disposal unit).

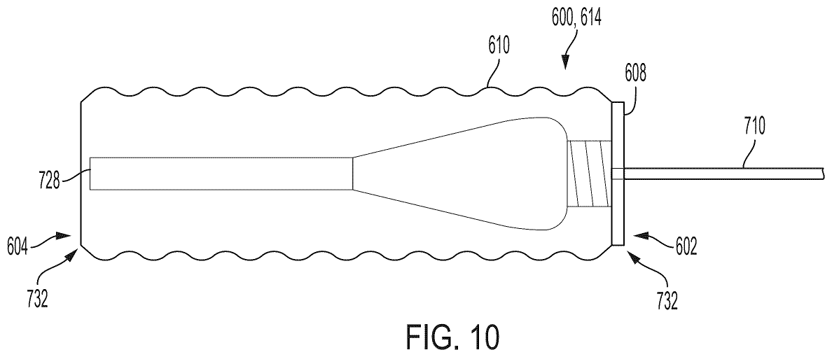

Referring to FIGS. 6-10, the radiation containment component 600 may include a proximal end 602 and a distal end 604 disposed opposite the proximal end 602. The proximal end 602 may be configured to connect to and cover a distal portion 712 of the delivery line connector 710 of the particulate delivery device 500. The delivery line connector 710 may be configured to receive the mixed particulate from the particulate delivery device 500. As shown in FIG. 10 described in greater detail further below, the distal end 604 may be configured to be disposed over and contain the microcatheter 720 connected to the delivery line connector 710 after use. Thus, the distal end 604 may be configured to seal together to contain the microcatheter 720 prior to disposal of, as a non-limiting example, the particulate delivery device 500 after use.

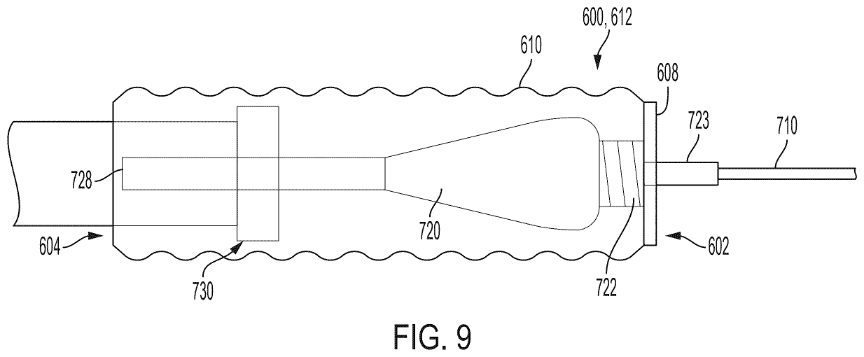

Referring to FIG. 6, showing the radiation containment component 600 in a folded position 606, the proximal end 602 in embodiments includes a stiff material 608, the distal end 604 includes a flexible material 610, and the flexible material 610 includes more elasticity than the stiff material 608. The flexible material 610 may include a deformable plastic, spring loaded material, or other suitable flexible material configured to roll, unroll, stretch, and/or return to a naturally biased position. The stiff material 608 may include cardboard, plastic, metal, or combinations thereof. The stiff material 608 may be a square, circular, or other shape from which the flexible material 610 extends. The flexible material 610 may be a low density material and include a thickness sufficient to assist with containment of radiation, such as, and not limited to, in a range of between 1 mm to 10 mm, such as up to 9 mm to block beta radiation, contain radiation, and thus provide additional shielding. The flexible material 610 may include a low density material to block beta radiation, the low density material comprising plastic, water or other fluid-filled materials, cloth, or other suitable flexible material, In embodiments, the flexible material 610 includes an accordion rib structure in a relaxed form and more elasticity than the stiff material 608 and is configured to stretch to a stretched form (e.g., an unfolded position 612 as shown in FIG. 9 or a sealed position 614 for disposal as shown in FIG. 10) to smooth the accordion rib structure and to cover and contain the microcatheter 720 prior to sealing of the distal end 604 of the radiation containment component 600. In embodiments, the flexible material 610 may be configured to unroll from a rolled form to cover and contain the microcatheter 720 prior to sealing of the distal end 604 of the radiation containment component 600. The distal end 604 may include sealing via an adhesive, fastening mechanism, or combinations thereof to seal together to contain the microcatheter 720 prior to disposal as described herein.

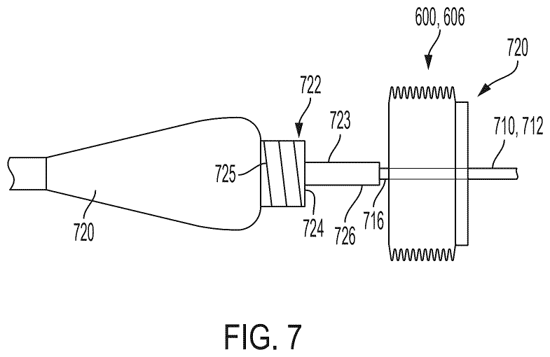

Referring to FIG. 7, the radiation containment component 600 in the folded position 606 is shown as disposed on the delivery line connector 710 of the particulate delivery device 500. The stiff material 608 of the proximal end 602 is configured to be disposed against an adjacent lip on the distal portion 712 of the delivery line connector 710 to prevent the proximal end 602 from moving in a distal direction toward the distal end 604. In embodiments, the stiff material 608 may include one or more engagement features to engagement with one or more corresponding engagement features of the adjacent lip on the distal portion 712 of the delivery line connector 710 such as one or more protrusions and/or bores configured to respectively engage with one or more respective other bores and/or protrusions or other locking mechanisms. As shown in FIG. 7-10, a distal end 716 of the delivery line connector 710 may be configured to connect to a proximal end 726 of the microcatheter 720. The distal end 716 of the delivery line connector 710 may be accessible when the distal end 604 of the radiation containment component 600 is unsealed. In embodiments, and as described below with respect to at least FIGS. 9 and 10, the distal end 604 of the radiation containment component 600 may be configured to cover the distal end 716 of the delivery line connector 710 when the distal end 604 of the radiation containment component 600 is sealed to contain the microcatheter 720 (such as at sealed ends 732).

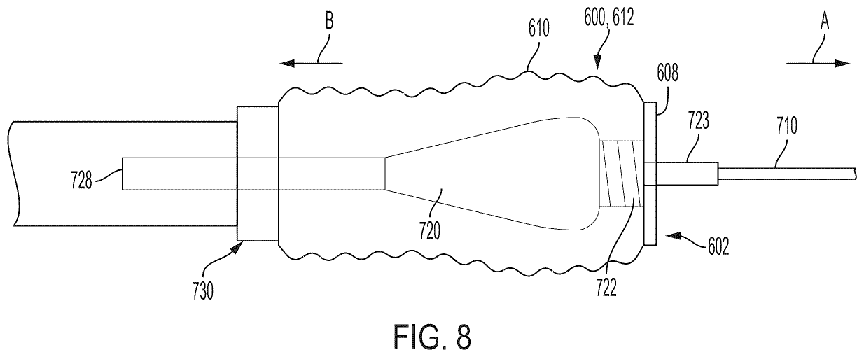

Referring again to FIG. 7, the microcatheter 720 may include as shown proximal end 726 including a fastener 722. The fastener 722 may be, as a non-limiting example, a luer connector or other fastening mechanism and may include as shown a tip 723, a ledge 724, and base 725 having a greater width than a width of the tip 723, the ledge 724 defined between the tip 723 and the base 725. The distal end 728 of the microcatheter 720 is shown in FIG. 8.

FIG. 8 illustrates the radiation containment component 600 in an unfolded position 612 that may be in a first unfolded state being distally pulled over the microcatheter 720 and approaching a base sheath 730 receiving the distal end 728 of the microcatheter 720. The microcatheter 720 may be configured to connect to a base connector such as the base sheath 730 for use to deliver the mixed particulate. Further, the microcatheter 720 may be configured to disconnect from the base connector such as the base sheath 730 after use and prior to the distal end 604 of the radiation containment component 600 containing the microcatheter 720 being sealed (as shown in FIG. 10).

Referring again to FIG. 8, in the first unfolded state, the fastener 722 of the microcatheter 720 is moved proximally toward proximal end 602 of the radiation containment component 600 in the direction of arrow A until the ledge 724 abuts the stiff material 608 of the radiation containment component 600. The abutment of the ledge 724 and the stiff material 608 aids to prevent the distal movement of the proximal end 602 of the radiation containment component 600. The tip 723 of the fastener 722 is shown in FIG. 8 when the ledge 724 abuts the stiff material 508 to extend through a bore in the stiff material 608 of the proximal end 602 of the radiation containment 600.

Further, the distal end 604 of the radiation containment component 600 is distally pulled in the direction of arrow B toward a base sheath 730 that has received the distal end 728 of microcatheter 720 for delivery of the mixed particulate from the particulate delivery device 500 to the patient, for example. The distal end 604 of the radiation containment component 600 may continue to be distally pulled in the direction of arrow B toward a base sheath 730 that has received the distal end 728 of microcatheter 720 to arrive at the position of FIG. 9.

FIG. 9 illustrates the radiation containment component 600 in the unfolded position 612 in a second unfolded state further distally pulled over the microcatheter 720 and the base sheath 730 receiving the distal end 728 of the microcatheter 720. The distal end 604 of the radiation containment component 600 may be sealed (and have, for example, a sealed end 732 as also shown in FIG. 10) prior to use of the connected microcatheter 720 and the delivery line connector 710 to deliver the mixed particulate to the patient.

FIG. 10 illustrates the radiation containment component 600 that is unfolded and in a sealed position 614 for disposal (as, for example, hazardous waste) while being disposed and sealed over the distal end 728 of the microcatheter 720 removed from the base sheath 730. Such sealing is shown as sealed ends 732. In embodiments, after use, the distal end 728 of the microcatheter 720 may be detached from the base sheath 730 and the distal end 604 of the radiation containment component 600 may be sealed via a sealed end 732 over the distal end 728 of the microcatheter 720. The proximal end 726 of the microcatheter 720 may still be connected to a distal end 716 of the delivery line connector 710, and the proximal end 602 of the radiation containment component 600 may continue to be sealed via a sealed end 732 over the distal portion 712 of the delivery line connector 710. In embodiments, the entire used particulate delivery device 500 may then be disposed of (such as in a hazardous waste disposal) including the radiation containment component 600 sealingly disposed via sealed ends 732 about the distal end 728 of the microcatheter 720 and the distal portion 712 of the delivery line connector 710.

In embodiments, a radiation sealing assembly for sealing and disposal may include the particulate delivery device 500 including the delivery line connector 710, the base connector such as the base sheath 730, the microcatheter 720, and the radiation containment component 600 as described herein. The microcatheter 720 may be used to deliver the mixed particulate from the particulate delivery device 500 and be configured to be connected to the base connector (e.g., the base sheath 730) and the delivery line connector 710 to deliver the mixed particulate. The microcatheter 720 may be configured to be disconnected from the base connector and connected to the delivery line connector 710 after use.

A method for sealing and disposal of the microcatheter 720 used to deliver the mixed particulate from the particulate delivery device 500 may include connecting the delivery line connector 710 of the particulate delivery device 500 to the microcatheter 720, such as shown in FIG. 7. The microcatheter 720 may be connected to the base connector (such as the base sheath 730 as shown in FIGS. 8 and 9). Further, the e mixed particulate may be delivered from the particulate delivery device 500 through the microcatheter 720 and base connector (e.g., the base sheath 730 of FIG. 9). The microcatheter 720 may be disconnected from the base connector after use, as shown in FIG. 10. The proximal end 602 of the radiation containment component 600 may be disposed over the distal portion 712 of the delivery line connector 710 of the particulate delivery device 500 such that the proximal end 602 does not move in a distal direction toward the distal end 604. The distal end 604 of the radiation containment component 600 may be extended over to contain the microcatheter 720 connected to the delivery line connector 710 after use. The distal end 604 of the radiation containment component 600 may be sealed together to contain the microcatheter 720 prior to disposal. In embodiments, and as shown in FIG. 10, the microcatheter 720 contained in the radiation containment component 600 and the particulate delivery device 500 connected to the microcatheter 720 and radiation containment component 600 may be disposed in a biohazardous waste disposal.

### III. Aspects Listing

Aspect 1. A radiation containment component for sealing a microcatheter used to deliver a mixed particulate from a particulate delivery device for disposal comprises a proximal end and a distal end disposed opposite the proximal end. The proximal end is configured to connect to and cover a distal portion of a delivery line connector of the particulate delivery device, the delivery line connector configured to receive the mixed particulate from the particulate delivery device. The distal end is configured to be disposed over and contain the microcatheter connected to the delivery line connector after use, and the distal end is configured to seal together to contain the microcatheter prior to disposal.

Aspect 2. The radiation containment component of Aspect 1, wherein the proximal end comprises a stiff material, the distal end comprises a flexible material, and the flexible material comprises more elasticity than the stiff material.

Aspect 3. The radiation containment component of Aspect 2, wherein the stiff material of the proximal end is configured to be disposed against an adjacent lip on the distal portion of the delivery line connector to prevent the proximal end from moving in a distal direction.

Aspect 4. The radiation containment component of any of Aspect 1 to Aspect 2, wherein the stiff material comprises cardboard, plastic, metal, or combinations thereof, and wherein the flexible material comprises a low density material, the low density material comprising a thickness of up to 9 mm to block beta radiation.

Aspect 5. The radiation containment component of any of Aspect 1 to Aspect 4, wherein the distal end of the radiation containment component is configured to cover the distal end of a delivery line connector when the distal end of the radiation containment component is sealed to contain the microcatheter, the distal end of the delivery line connector configured to connect to a proximal end of the microcatheter.

Aspect 6. The radiation containment component of Aspect 5, wherein the distal end of the delivery line connector is accessible when the distal end of the radiation containment component is unsealed.

Aspect 7. The radiation containment component of any of Aspect 1 to Aspect 6, wherein the proximal end comprises a stiff material, the distal end comprises a flexible material, and the flexible material comprises an accordion rib structure in a relaxed form and more elasticity than the stiff material and is configured to stretch to a stretched form to smooth the accordion rib structure and to cover and contain the microcatheter prior to sealing of the distal end.

Aspect 8. The radiation containment component of Aspect 1 to Aspect 7, wherein the distal end comprises an adhesive, fastening mechanism, or combinations thereof to seal together to contain the microcatheter prior to disposal.

Aspect 9. The radiation containment component of any of Aspect 1 to Aspect 8, wherein the microcatheter is configured to connect to a base connector for use to deliver the mixed particulate, and the microcatheter is configured to disconnect from the base connector after use and prior to the distal end containing the microcatheter being sealed.

Aspect 10. A radiation sealing assembly for sealing and disposal comprising a particulate delivery device comprising a delivery line connector, a base connector, a microcatheter, and a radiation containment component. The microcatheter is used to deliver a mixed particulate from the particulate delivery device, the microcatheter configured to be connected to the base connector and the delivery line connector to deliver the mixed particulate, and the microcatheter configured to be disconnected from the base connector and connected to the delivery line connector after use. The radiation containment component comprises a proximal end and a distal end disposed opposite the proximal end. The proximal end is configured to connect to and cover a distal portion of the delivery line connector of the particulate delivery device, the delivery line connector configured to receive the mixed particulate from the particulate delivery device. The distal end is configured to be disposed over and contain the microcatheter connected to the delivery line connector after use. The distal end is configured to seal together to contain the microcatheter prior to disposal.

Aspect 11. The radiation sealing assembly of Aspect 10, wherein the proximal end comprises a stiff material, the distal end comprises a flexible material, and the flexible material comprises more elasticity than the stiff material.

Aspect 12. The radiation sealing assembly of Aspect 11, wherein the stiff material of the proximal end is configured to be disposed against an adjacent lip on the distal portion of the delivery line connector to prevent the proximal end from moving in a distal direction.

Aspect 13. The radiation sealing assembly of any of Aspect 10 to Aspect 12, wherein the stiff material comprises cardboard, plastic, metal, or combinations thereof.

Aspect 14. The radiation sealing assembly of any of Aspect 10 to Aspect 13, wherein the distal end of the radiation containment component is configured to cover a distal end of the delivery line connector when the distal end of the radiation containment component is sealed to contain the microcatheter, the distal end of the delivery line connector configured to connected to a proximal end of the microcatheter.

Aspect 15. The radiation sealing assembly of Aspect 14, wherein the distal end of the delivery line connector is accessible when the distal end of the radiation containment component is unsealed.

Aspect 16. The radiation sealing assembly of any of Aspect 10 to Aspect 15, wherein the proximal end comprises a stiff material, the distal end comprises a flexible material, and the flexible material comprises an accordion rib structure in a relaxed form and more elasticity than the stiff material and is configured to stretch to a stretched form to smooth the accordion rib structure and to cover and contain the microcatheter prior to sealing of the distal end.

Aspect 17. The radiation sealing assembly of any of Aspect 10 to Aspect 16, wherein the distal end comprises an adhesive, fastening mechanism, or combinations thereof to seal together to contain the microcatheter prior to disposal.

Aspect 18. The radiation sealing assembly of any of Aspect 10 to Aspect 17, wherein the microcatheter is configured to connect to a base connector for use to deliver the mixed particulate, and the microcatheter is configured to disconnect from the base connector after use and prior to the distal end containing the microcatheter and being sealed.

Aspect 19. A method for sealing and disposal of a microcatheter used to deliver a mixed particulate from a particulate delivery device comprising connecting a delivery line connector of the particulate delivery device to the microcatheter, connecting the microcatheter to a base connector, delivering the mixed particulate from the particulate delivery device through the microcatheter and the base connector, and disconnecting the microcatheter from the base connector after use. The method further comprises disposing a proximal end of a radiation containment component over a distal portion of the delivery line connector of the particulate delivery device such that the proximal end does not move in a distal direction, extending a distal end of the radiation containment component over to contain the microcatheter connected to the delivery line connector after use, and sealing the distal end together to contain the microcatheter prior to disposal.

Aspect 20. The method of Aspect 19, further comprising disposing the microcatheter contained in the radiation containment component and the particulate delivery device connected to the microcatheter and the radiation containment component in a biohazardous waste disposal.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

For the purposes of describing and defining the present disclosure it is noted that the term “substantially” is used herein to represent the inherent degree of uncertainty that may be attributed to any quantitative comparison, value, measurement, or other representation. The term “substantially” is used herein also to represent the degree by which a quantitative representation may vary from a stated reference without resulting in a change in the basic function of the subject matter at issue. As such, it is used to represent the inherent degree of uncertainty that may be attributed to any quantitative comparison, value, measurement, or other representation, referring to an arrangement of elements or features that, while in theory would be expected to exhibit exact correspondence or behavior, may in practice embody something slightly less than exact.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. A radiation containment component for sealing a microcatheter used to deliver a mixed particulate from a particulate delivery device for disposal, the radiation containment component comprising:
a proximal end and a distal end disposed opposite the proximal end;
the proximal end configured to connect to and cover a distal portion of a delivery line connector of the particulate delivery device, the delivery line connector configured to receive the mixed particulate from the particulate delivery device;
the distal end configured to be disposed over and contain the microcatheter connected to the delivery line connector after use; and
the distal end configured to seal together to contain the microcatheter prior to disposal.

2. The radiation containment component of claim 1, wherein the proximal end comprises a stiff material, the distal end comprises a flexible material, and the flexible material comprises more elasticity than the stiff material.

3. The radiation containment component of claim 2, wherein the stiff material of the proximal end is configured to be disposed against an adjacent lip on the distal portion of the delivery line connector to prevent the proximal end from moving in a distal direction.

4. The radiation containment component of claim 2, wherein the stiff material comprises cardboard, plastic, metal, or combinations thereof, and wherein the flexible material comprises a low density material, the low density material comprising a thickness of up to 9 mm to block beta radiation.

5. The radiation containment component of claim 1, wherein the distal end of the radiation containment component is configured to cover a distal end of the delivery line connector when the distal end of the radiation containment component is sealed to contain the microcatheter, the distal end of the delivery line connector configured to connect to a proximal end of the microcatheter.

6. The radiation containment component of claim 5, wherein the distal end of the delivery line connector is accessible when the distal end of the radiation containment component is unsealed.

7. The radiation containment component of claim 1, wherein the proximal end comprises a stiff material, the distal end comprises a flexible material, and the flexible material comprises an accordion rib structure in a relaxed form and more elasticity than the stiff material and is configured to stretch to a stretched form to smooth the accordion rib structure and to cover and contain the microcatheter prior to sealing of the distal end.

8. The radiation containment component of claim 1, wherein the distal end comprises an adhesive, fastening mechanism, or combinations thereof to seal together to contain the microcatheter prior to disposal.

9. The radiation containment component of claim 1, wherein the microcatheter is configured to connect to a base connector for use to deliver the mixed particulate, and the microcatheter is configured to disconnect from the base connector after use and prior to the distal end containing the microcatheter being sealed.

10. A radiation sealing assembly for sealing and disposal, the radiation sealing assembly comprising:
a particulate delivery device comprising a delivery line connector;
a base connector;
a microcatheter used to deliver a mixed particulate from the particulate delivery device, the microcatheter configured to be connected to the base connector and the delivery line connector to deliver the mixed particulate, and the microcatheter configured to be disconnected from the base connector and connected to the delivery line connector after use; and
a radiation containment component comprising:
a proximal end and a distal end disposed opposite the proximal end;
the proximal end configured to connect to and cover a distal portion of the delivery line connector of the particulate delivery device, the delivery line connector configured to receive the mixed particulate from the particulate delivery device;
the distal end configured to be disposed over and contain the microcatheter connected to the delivery line connector after use; and
the distal end configured to seal together to contain the microcatheter prior to disposal.

11. The radiation sealing assembly of claim 10, wherein the proximal end comprises a stiff material, the distal end comprises a flexible material, and the flexible material comprises more elasticity than the stiff material.

12. The radiation sealing assembly of claim 11, wherein the stiff material of the proximal end is configured to be disposed against an adjacent lip on the distal portion of the delivery line connector to prevent the proximal end from moving in a distal direction.

13. The radiation sealing assembly of claim 11, wherein the stiff material comprises cardboard, plastic, metal, or combinations thereof.

14. The radiation sealing assembly of claim 11, wherein the distal end of the radiation containment component is configured to cover a distal end of the delivery line connector when the distal end of the radiation containment component is sealed to contain the microcatheter, the distal end of the delivery line connector configured to connected to a proximal end of the microcatheter.

15. The radiation sealing assembly of claim 14, wherein the distal end of the delivery line connector is accessible when the distal end of the radiation containment component is unsealed.

16. The radiation sealing assembly of claim 10, wherein the proximal end comprises a stiff material, the distal end comprises a flexible material, and the flexible material comprises an accordion rib structure in a relaxed form and more elasticity than the stiff material and is configured to stretch to a stretched form to smooth the accordion rib structure and to cover and contain the microcatheter prior to sealing of the distal end.

17. The radiation sealing assembly of claim 10, wherein the distal end comprises an adhesive, fastening mechanism, or combinations thereof to seal together to contain the microcatheter prior to disposal.

18. The radiation sealing assembly of claim 10, wherein the microcatheter is configured to connect to the base connector for use to deliver the mixed particulate, and the microcatheter is configured to disconnect from the base connector after use and prior to the distal end containing the microcatheter and being sealed.

19. A method for sealing and disposal of a microcatheter used to deliver a mixed particulate from a particulate delivery device, the method comprising:
connecting a delivery line connector of the particulate delivery device to the microcatheter;
connecting the microcatheter to a base connector;
delivering the mixed particulate from the particulate delivery device through the microcatheter and the base connector;
disconnecting the microcatheter from the base connector after use;
disposing a proximal end of a radiation containment component over a distal portion of the delivery line connector of the particulate delivery device such that the proximal end does not move in a distal direction;
extending a distal end of the radiation containment component over to contain the microcatheter connected to the delivery line connector after use; and
sealing the distal end together to contain the microcatheter prior to disposal.

20. The method of claim 19, further comprising disposing the microcatheter contained in the radiation containment component and the particulate delivery device connected to the microcatheter and the radiation containment component in a biohazardous waste disposal.
